(19) United States
(12) Patent Application Publication
(10) Pub. No.: US 2018/0256389 A1
(43) Pub. Date: Sep. 13, 2018

(54) PELVIC ORGAN SUPPORT DEVICES AND RELATED METHODS

(71) Applicant: CooperSurgical, Inc., Trumbull, CT (US)

(72) Inventors: Mona Asfar, Staten Island, NY (US); Karrison Connors, Tinton Falls, NJ (US); Deanna Quackenbush, Selden, NY (US); Carla Souza, Rahway, NJ (US); Nicole E. Miller, Bridgeport, CT (US); Rosemary Michelle Garofalo, North Haven, CT (US)

(21) Appl. No.: 15/864,138

(22) Filed: Jan. 8, 2018

Related U.S. Application Data
(60) Provisional application No. 62/469,856, filed on Mar. 10, 2017.

(51) Int. Cl. A61F 6/08

(52) U.S. Cl. CPC A61F 6/08

(57) ABSTRACT

A pelvic organ support device includes a base configured to support a pelvic organ and a stem extending from the base for manipulating the pelvic organ support device. The base includes an outer rim and a wall section that extends radially inward from the outer rim. A thickness of the wall section of the base remains constant or decreases along at least a portion of the wall section between the stem and the outer rim of the base.

### CROSS-REFERENCE TO RELATED APPLICATION

This application claims priority to U.S. Provisional Patent Application No. 62/469,856, filed on Mar. 10, 2017, the entire contents of which is incorporated herein by reference.

### TECHNICAL FIELD

This disclosure relates to pelvic organ support devices, and more particularly to flexible pelvic organ support devices that can be deployed and removed by a patient outside of a medical facility.

### BACKGROUND

Pelvic organ prolapse (POP) is characterized by descent of female genital organs beyond normal anatomical confines of the organs due to weakening of tension within muscles in the pelvic floor. POP typically occurs when the pelvic floor collapses as a result of childbirth or heavy lifting, which can tear soft tissues. A pessary is a non-surgical option for treating POP and associated issues (e.g., stress urinary incontinence). A pessary is used as a space-filling object capable of supporting the pelvic organs (e.g., the bladder, uterus, rectum, and small bowel) and for addressing vaginal vault prolapse. A pessary is inserted into the vaginal canal to elevate the compartment of the vagina that has dropped.

FIG. 1 is a cross-sectional view of a conventional gellhorn pessary 1000 that includes a support base 1002 for supporting pelvic organs and a stem 1004 for manipulating the pessary 1000. Over 50% of women suffering from pelvic organ prolapse and stress urinary incontinence in the United States do not use pessaries due to their inconvenience, lack of patient acceptance, discomfort, resulting vaginal bleeding or discharge, worsening urinary incontinence, poor fit, difficulty in insertion and removal, erosion of the vaginal wall, interference with sexual life, odor, and other side effects. Because current designs are difficult to maintain and require frequent physician intervention, roughly 30% of patients discontinue pessary use after six months or less and may ultimately turn to more invasive surgical options.

### SUMMARY

The present disclosure provides improved pessaries and associated delivery devices that can be used and maintained by patients outside of a medical facility (e.g., at home). The disclosed pessaries provide the necessary structural support of conventional gellhorn pessaries, but have less material than gellhorn pessaries, such that the disclosed pessaries are less stiff and more flexible than gellhorn pessaries. Owing to the increased flexibility of the disclosed pessaries, the disclosed pessaries can be more easily removed, thereby mitigating associated side effects and allowing for easier, more convenient, and more frequent cleaning at home, allowing for prevention of vaginal wall erosion, and allowing for removal for sexual intercourse. The disclosed pessaries may also be provided with a delivery device that aids in self-insertion. Pessary systems including the disclosed pessaries and associated delivery device alleviates the need to see a gynecologist and provides an easy, low cost solution to problems associated with pelvic organ prolapse (POP) and stress urinary incontinence, thereby reducing the need for expensive and high-risk surgeries.

In one aspect, a pelvic organ support device includes a base configured to support a pelvic organ and a stem extending from the base for manipulating the pelvic organ support device. The base includes an outer rim and a wall section that extends radially inward from the outer rim. A thickness of the wall section of the base remains constant or decreases along at least a portion of the wall section between the stem and the outer rim of the base.

In some embodiments, the base defines an inner surface that is generally shaped as a stepped cone.

In certain embodiments, the base defines multiple alternating negative sections and positive material sections along an outer surface of the base.

In some embodiments, the base defines an inner surface that is generally conical shaped.

In certain embodiments, the wall section of the base is spaced apart from an edge of the outer rim.

In some embodiments, the wall section of the base is aligned with an edge of the outer rim. In certain embodiments, a force ranging from about 15 N to about 20 N is needed to bend the stem to a point of contact with the base.

In another aspect, a pelvic organ support system includes the pelvic organ support device and a tubular applicator sized to surround the pelvic organ support device.

### DESCRIPTION OF DRAWINGS

FIG. 1 is a cross-sectional view of a conventional pessary.

FIG. 2 is a perspective view of an improved pessary.

FIG. 3 is a cross-sectional view of the improved pessary of FIG. 2.

FIG. 4 is a perspective view of an improved pessary.

FIG. 5 is a cross-sectional view of the improved pessary of FIG. 4.

FIG. 6 is a perspective view of an improved pessary.

FIG. 7 is a cross-sectional view of the improved pessary of FIG. 6.

FIG. 8 is a cross-sectional view of the improved pessary of any of FIGS. 2-7 positioned within a patient.

FIG. 9 is a side view of the improved pessary of any of FIGS. 2-7 positioned within a delivery device.

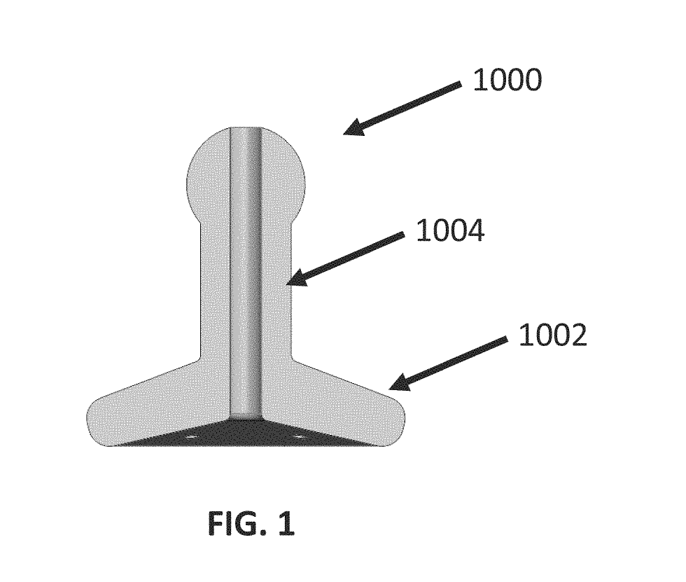

Like reference symbols in the various figures indicate like elements. In some examples, illustrations shown in the drawings may not be drawn to scale.

### DETAILED DESCRIPTION

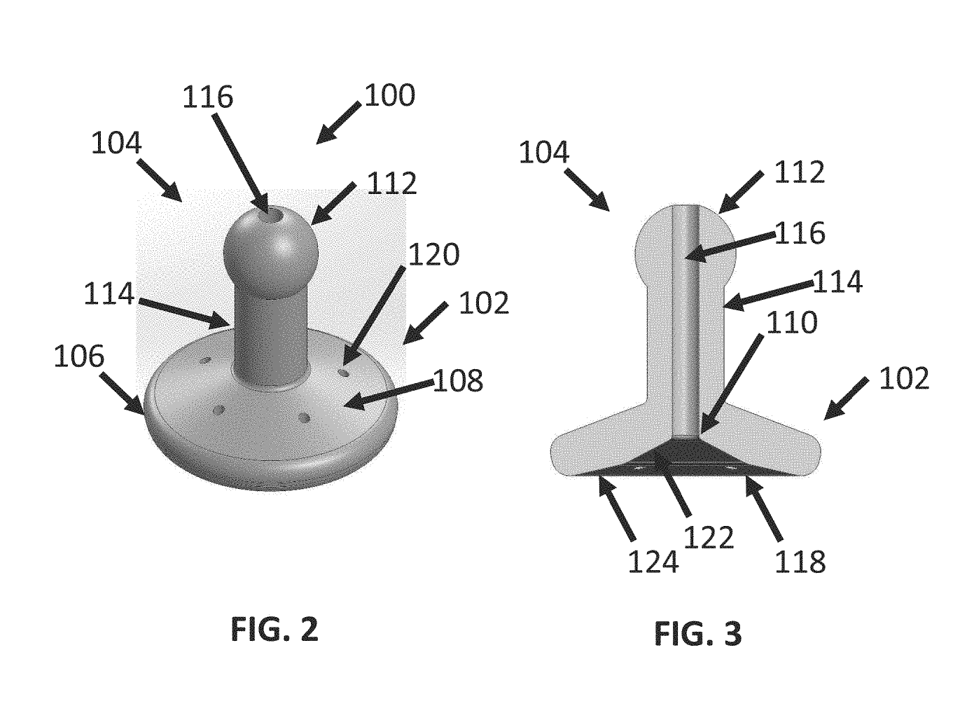

FIGS. 2 and 3 are perspective and cross-sectional views, respectively, of an improved pessary 100 used for treating pelvic organ prolapse (POP). The pessary 100 includes a support base 102 designed to support pelvic organs within a patient and a stem 104 extending from the support base 102 for manipulation of the pessary 100. The stem 104 includes a knob 112 and a shaft 114 that together define a central channel 116 through which fluid can flow into the vagina.

The support base 102 includes an outer rim 106 (e.g., an outer ring) that is sized to surround the cervix (e.g., the vaginal fornices) and a floor 108 that extends radially from the stem 104 to the outer rim 106. The floor 108 is an annular shaped, disk-like wall that extends at a variable angle from an end 110 of the central channel 116 to the outer rim 106 to define a receptacle surface 118 for the cervix. The floor 108 defines multiple through channels 120 that allow passage of fluid into the vagina. As compared to the base 1002 of the gellhorn pessary 1000, the base 102 of the pessary 100 has less material, such that the pessary 100 is less stiff and more flexible than the pessary 1000 between the base 102 and the stem 104. For example, the floor 108 has a first reduced material section 122 that extends from the end 110 of the central channel 116 to a second material section 124 that reflects a corresponding profile of the base 1002 of the gellhorn pessary 1000. As a result, the receptacle surface 118 of the base 102 is generally shaped as a stepped cone. Owing to the increased flexibility of the pessary 100, the pessary 100 has an increased bending capability as compared to the pessary 1000, such that the pessary 100 is easier to insert into and position within the vaginal canal than the pessary 1000.

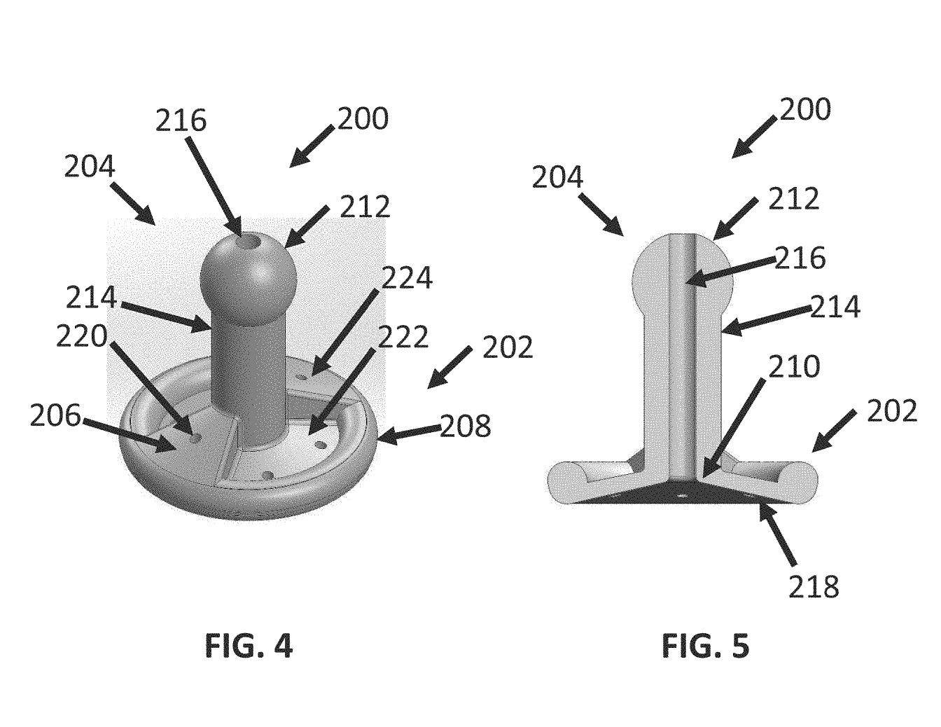

FIGS. 4 and 5 are perspective and cross-sectional views, respectively, of an improved pessary 200 used for POP. The pessary 200 includes a support base 202 designed to support pelvic organs within a patient and a stem 204 extending from the support base 202 for manipulation of the pessary 200. The stem 204 includes a knob 212 and a shaft 214 that together define a central channel 216 through which fluid can flow into the vagina.

The support base 202 includes an outer rim 206 (e.g., an outer ring) that is sized to surround the cervix (e.g., the vaginal fornices) and a floor 208 that extends radially from the stem 204 to the outer rim 206. The floor 208 is a generally annular shaped, disk-like wall that extends at an angle from an end 210 of the central channel 216 to the outer rim 206 to define a receptacle surface 218 for the cervix. The floor 208 defines multiple through channels 220 that allow passage of fluid into the vagina. As compared to the base 1002 of the gellhorn pessary 1000, the base 202 of the pessary 200 has less material, such that the pessary 200 is less stiff and more flexible than the pessary 1000 between the base 202 and the stem 204. For example, the floor 208 has two arc-shaped negative (e.g., reduced material) sections 222 located along a top surface of the base 202 and on opposite sides of the stem 204. The negative sections 222 define two positive material sections 224 located along the top surface of the base 202 and on opposite sides of the stem 204. The positive material sections 224 reflect a corresponding profile of the base 1002 of the gellhorn pessary 1000. Owing to the increased flexibility of the pessary 200, the pessary 200 has an increased bending capability as compared to the pessary 1000, such that the pessary 200 is easier to insert into and position within the vaginal canal than the pessary 1000.

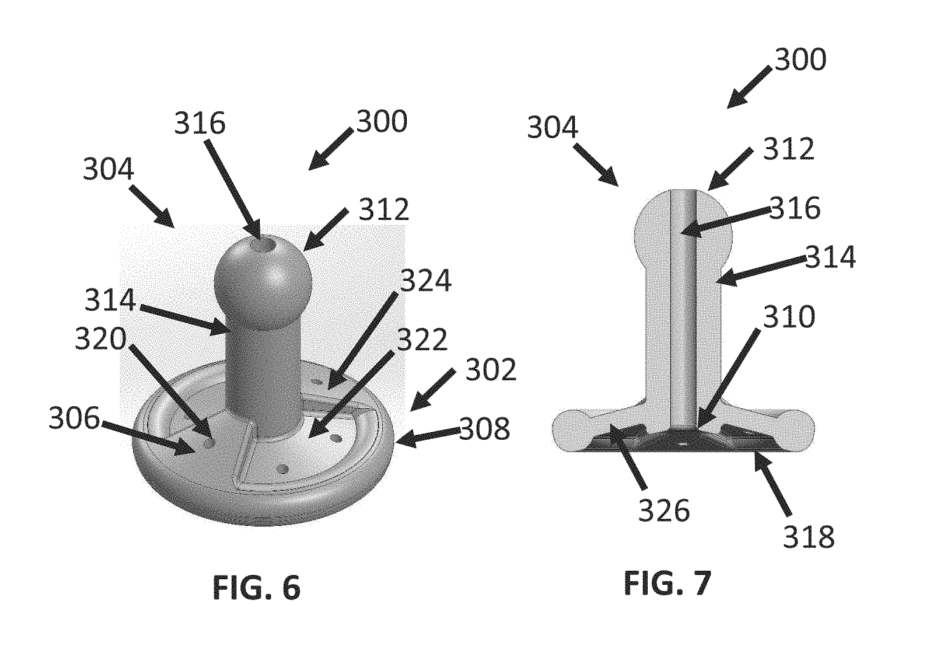

In some embodiments, a pessary that is similar in construction and function to the pessary 200 may include a different number of negative and positive sections or negative and positive sections with shapes different from those shown in FIG. 4. FIGS. 6 and 7 are perspective and cross-sectional views, respectively, of an improved pessary 300 used for POP. The pessary 300 includes a support base 302 designed to support pelvic organs within a patient and a stem 304 extending from the support base 302 for manipulation of the pessary 300. The stem 304 includes a knob 312 and a shaft 314 that together define a central channel 316 through which fluid can flow into the vagina.

The support base 302 includes an outer rim 306 (e.g., an outer ring) that is sized to surround the cervix (e.g., the vaginal fornices) and a floor 308 that extends radially from the stem 304 to the outer rim 306. The floor 308 is a generally annular shaped, disk-like wall that extends at an angle from an end 310 of the central channel 316 to the outer rim 306 to define a receptacle surface 318 for the cervix. The floor 308 defines multiple through channels 320 that allow passage of fluid into the vagina. As compared to the base 1002 of the gellhorn pessary 1000, the base 302 of the pessary 300 has less material, such that the pessary 300 is less stiff and more flexible than the pessary 1000 between the base 302 and the stem 304. For example, the floor 308 has two arc-shaped negative (e.g., reduced material) sections 322 located along a surface of the base 302 and on opposite sides of the stem 304. The negative sections 322 are less shallow (e.g., have a smaller height) than the negative sections 222 of the pessary 200. The negative sections 322 define two positive material sections 324 located along the same surface of the base 302 and on opposite sides of the stem 304. The positive material sections 324 reflect a corresponding profile of the base 1002 of the gellhorn pessary 1000. Additionally, the floor 308 has a reduced material section 326 that extends from the end 310 of the central channel 316 to the outer rim 306. Accordingly, the receptacle surface 318 defines a larger volume than that of the receptacle surface 118 of the pessary 100. Owing to the increased flexibility of the pessary 300, the pessary 300 has an increased bending capability as compared to the pessary 1000, such that the pessary 300 is easier to insert into and position within the vaginal canal than the pessary 1000.

In some embodiments, a pessary that is similar in construction and function to the pessary 300 may include a different number of negative and positive sections or negative and positive sections with shapes different from those shown in FIG. 6. The increased flexibility of the pessaries 100, 200, 300 does not compromise their functional capability to support the pelvic organs when positioned within a patient. In some embodiments, the pessaries 100, 200, 300 have a total length ranging from about 5.2 cm to about 10.2 cm and a base diameter of about 4.4 cm such that the pessaries 100, 200, 300 are sized to accommodate about 85% of the patient population. In some embodiments, the pessaries 100, 200, 300 are coated with a lubricant that can ease insertion of the pessaries 100, 200, 300 into a delivery device or within the vaginal canal. However, any of the pessaries 100, 200, 300 may be modified in one or more dimensions to accommodate patients that have larger or smaller anatomical features. In some embodiments, the pessaries 100, 200, 300 are made of a 60 durometer rubber-like material. In some embodiments, the pessaries 100, 200, 300 may be made of a different material with a different hardness. In some embodiments, the pessaries 100, 200, 300 are formed as a single, unitary structure using 3D printing. In some embodiments, the pessaries 100, 200, 300 may be manufactured via a different technique or formed from two or more separate components that are joined together.

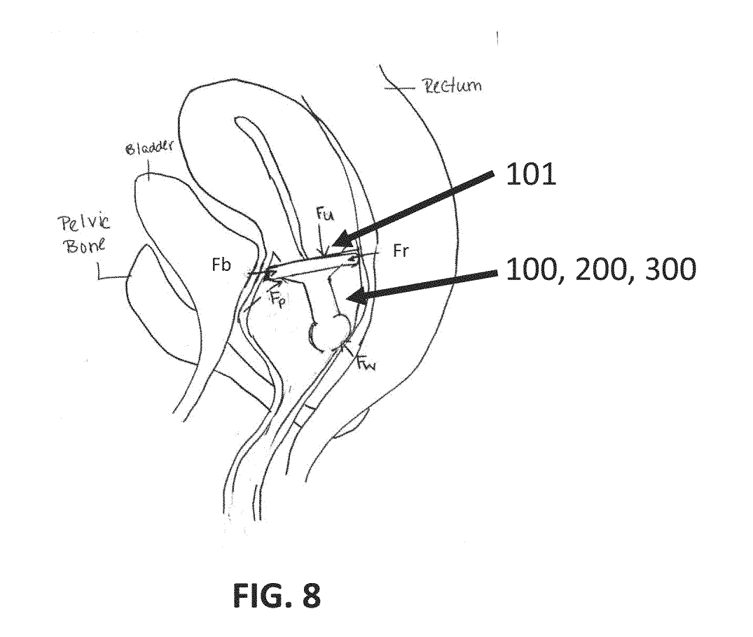

FIG. 8 is a cross-sectional view of any of the pessaries 100, 200, 300 positioned within a patient. The pessary 100, 200, 300 may be positioned against the vaginal fornices 101 and may be held in place by forces exerted from the uterus (Fᵤ), the bladder (F_{b}), the rectum (Fᵣ), the anterior vaginal wall (Fᵣ), and the posterior vaginal wall (F_{w}).

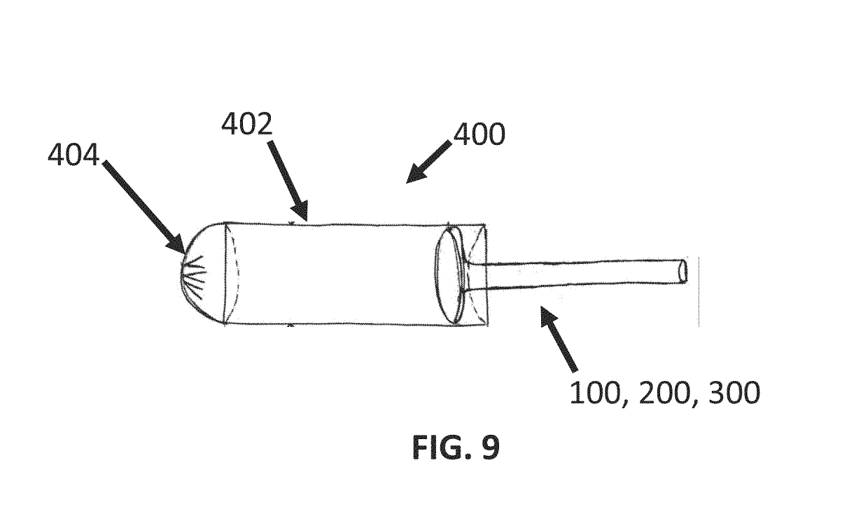

FIG. 9 illustrates a side view of any of the pessaries 100, 200, 300 positioned within a delivery device 400 (e.g., an applicator) that can facilitate insertion of the pessary 100, 200, 300 into the vagina. The delivery device 400 includes an elongate sheath 402 sized to accommodate the pessaries 100, 200, 300 and a rounded end section 404 sized to accommodate the bases 102, 202, 302 of the pessaries 100, 200, 300. The elongate sheath 402 has a length of about 11.4 cm, such that the delivery device 400 can completely surround any of the pessaries 100, 200, 300. Any of the pessaries 100, 200, 300 may be positioned within the delivery device 400 in a folded state and moved from the delivery device 400 into position within the patient with a low, manageable deployment force. The delivery device 400 allows a patient to insert any of the pessaries 100, 200, 300 without assistance from a physician.

Each of the pessaries 100, 200, 300 underwent a bending force test to determine the force required to bend the stem 104, 204, 304 to the base 102, 202, 302. After securing the pessary 100, 200, 300 to a mount at an angle of 45 degrees, a hand held force gauge was used to bend the stem 104, 204, 304 of pessary 100, 200, 300 to the base 102, 202, 302. The force required for bending was measured and recorded. This process was repeated 10 times on the pessaries 100, 200, 300, as well as on the gellhorn pessary 1000 to ensure an adequate statistical sample. Following data collection, a one-way ANOVA test was performed on the average force required to bend the stem to the base of the pessary on each sample. The average bending force was computed for each pessary 100, 200, 300 and the pessary 1000. Testing showed that the average force required to bend the stem to the base of the pessaries was 25 N for the pessary 1000, 20 N for the pessary 100, 17 N for the pessary 200, and 15 N for the pessary 300. The lower bending forces of the pessaries 100, 200, 300 can ease patient self-insertion and self-removal of the pessaries 100, 200, 300, as compared to the pessary 1000.

A number of embodiments have been described. Nevertheless, it will be understood that various modifications may be made without departing from the spirit and scope of the present disclosure. Accordingly, other embodiments are within the scope of the following claims.

What is claimed is:

1. A pelvic organ support device, comprising:
a base configured to support a pelvic organ, the base comprising an outer rim and a wall section that extends radially inward from the outer rim; and
a stem extending from the base for manipulating the pelvic organ support device,
wherein a thickness of the wall section of the base remains constant or decreases along at least a portion of the wall section between the stem and the outer rim of the base.

2. The pelvic organ support device of claim 1, wherein the base defines an inner surface that is generally shaped as a stepped cone.

3. The pelvic organ support device of claim 1, wherein the base defines a plurality of alternating negative sections and positive material sections along an outer surface of the base.

4. The pelvic organ support device of claim 1, wherein the base defines an inner surface that is generally conical shaped.

5. The pelvic organ support device of claim 1, wherein the wall section of the base is spaced apart from an edge of the outer rim.

6. The pelvic organ support device of claim 1, wherein the wall section of the base is aligned with an edge of the outer rim.

7. The pelvic organ support device of claim 1, wherein a force ranging from about 15 N to about 20 N is needed to bend the stem to a point of contact with the base.

8. A pelvic organ support system, comprising:
a pelvic organ support device, comprising:
a base configured to support a pelvic organ, the base comprising an outer rim and a wall section that extends radially inward from the outer rim,
a stem extending from the base for manipulating the pelvic organ support device,
wherein a thickness of the wall section of the base remains constant or decreases along at least a portion of the wall section between the stem and the outer rim of the base; and
a tubular applicator sized to surround the pelvic organ support device.
